(19) United States
(12) Patent Application Publication
(10) Pub. No.: US 2023/0282355 A1
(43) Pub. Date: Sep. 7, 2023

(54) Integrated Biomarker System for Evaluating Risks of Impaired Fasting Glucose (IFG) and Type 2 Diabetes Mellitus (T2DM)

(71) Applicant: BEIJING FRIENDSHIP HOSPITAL, CAPITAL MEDICAL UNIVERSITY, Beijing (CN)

(72) Inventors: Dan YAN, Beijing (CN); Jianglan LONG, Beijing (CN); Zhirui YANG, Beijing (CN)

(73) Assignee: BEIJING FRIENDSHIP HOSPITAL, CAPITAL MEDICAL UNIVERSITY, Beijing (CN)

(21) Appl. No.: 17/623,233

(22) PCT Filed: Apr. 26, 2021
(86) PCT No.: PCT/CN2021/089772

§ 371 Date: Dec. 27, 2021

(30) Foreign Application Priority Data
Feb. 3, 2021 (CN) 202110144115.8

(51) Int. Cl. G16H 50/30; G01N 33/68; G06N 3/08

(52) U.S. Cl. CPC G16H 50/30; G01N 33/6848; G06N 3/08; G01N 2800/042; G01N 2800/50

(57) ABSTRACT

An integrated biomarker system for evaluating a risk of impaired fasting glucose (IFG) and type 2 diabetes mellitus (T2DM) for the first time is disclosed. The integrated biomarker system includes quantitative determination results of L-glutamine, L-valine, L-leucine, L-lysine, L-proline, L-phenylalanine, L-arginine, L-glutamic acid, L-isoleucine, L-methionine, L-carnitine, acetyl-L-carnitine, lysophosphatidyl choline (LPC (P-16:0)), LPC (17:0), LPC (14:0) and propionyl-L-carnitine in a sample. The integrated biomarker system for IFG and T2DM of subject serum sample contains a correlative biomarker group on a biological network path to reflect the overall metabolic characteristics information of IFG and T2DM and to avoid that characteristic information of a disease cannot be reflected integrally and completely due to single or separate analysis of a biomarker.

### CROSS REFERENCE TO THE RELATED APPLICATIONS

This application is the national phase entry of International Application No. PCT/CN2021/089772, filed on Apr. 26, 2021, which is based upon and claims priority to Chinese Patent Application No. 202110144115.8, filed on Feb. 03, 2021, the entire contents of which are incorporated herein by reference.

### TECHNICAL FIELD

The present invention relates to the field of pharmaceutical determination, and in particular to an integrated biomarker system for evaluating a risk of impaired fasting glucose (IFG) and type 2 diabetes mellitus (T2DM).

### BACKGROUND

Type 2 diabetes mellitus (T2DM) is a kind of chronic metabolic disease; impaired fasting glucose (IFG) is a type of prediabetes, and the fasting blood glucose is between the normal value and T2DM. Generally, T2DM is an irreversible and lifelong disease, while IFG is reversible. The rate of converting IFG into diabetes mellitus may be reduced by strict diet control, more exercise and other lifestyle intervention. A national survey published in the The New England Journal of Medicine by professor Yang Wenying in 2007 shows that the number of diabetic patients in China has been nearly 100 million. Global Diabetes Reports issued by the World Health Organization in 2016 for the first time shows that about 500 millions of adults are in prediabetic phase, but the diagnostic rate of prediabetes is low, most people do not yet know they are in prediabetic phase. The diagnostic criterion of the World Health Organization on IFG and T2DM in 1999 is based on the definition of fasting blood glucose, but when the subject is about to develop into IFG or T2DM, the fasting blood-glucose has reduced diagnostic sensitivity. Therefore, it is crucial to explore a biomarker for the diagnostic sensitivity of IFG and T2DM, which is of great significance to the early diagnosis of IFG and T2DM, early intervention of IFG, prevention and control of T2DM.

Metabolite not only reflects the change of genome and proteome, but also is influenced by other factors, such as environmental factors and intestinal flora. Moreover, metabolite has stronger dynamics and thus, is more sensitive to the change reflection of an organism. Chinese patent CN104769434B discloses that metabolites glycine, lysophosphatidyl choline and acetyl carnitine C2 may be used for identifying a tendency of developing into T2DM in a subject. However, the biomarker for the diagnosis of IFG and T2DM presents an isolated and dispersed state. Most of the researches are based on the study of unicentral non-targeted metabonomics and thus, have low reproducibility, which is difficult to embody clinical application values of a biomarker. In terms of systems biology, there is a correlation among a plurality of metabolites. Therefore, it is of practical application value to serve a plurality of quantitative metabolites as a biomarker for the diagnosis of IFG and T2DM. An integrated biomarker system is a characteristic change spectrum formed by integrating biomarkers of a disease, and is a real synthetic response of a variation trend of in vivo important metabolites and bio-network association signals. However, no integrated biomarker system for IFG and T2DM patients have been studied and established up to now.

In view of this, the present invention is provided herein.

### SUMMARY

The present invention provides an integrated biomarker system for evaluating a risk of impaired fasting glucose (IFG) and type 2 diabetes mellitus (T2DM); the integrated biomarker system includes quantitative determination results of L-glutamine within a scope of 2,000-16,0000 ng/mL, L-valine within a scope of 1,200-96,000 ng/mL, L-leucine within a scope of 1,000-8,0000 ng/mL, L-lysine within a scope of 800-64,000 ng/mL, L-proline within a scope of 800-64,000 ng/mL, L-phenylalanine within a scope of 500-40,000 ng/mL, L-arginine within a scope of 500-40,000 ng/mL, L-glutamic acid within a scope of 500-40,000 ng/mL, L-isoleucine within a scope of 300-24,000 ng/mL, L-methionine within a scope of 250-20,000 ng/mL, L-carnitine within a scope of 200-16,000 ng/mL, acetyl-L-carnitine within a scope of 80-6,400 ng/mL, lysophosphatidyl choline (LPC (P-16:0)) within a scope of 60-4,800 ng/mL, LPC (17:0) within a scope of 60-4,800 ng/mL, LPC (14:0) within a scope of 40-3,200 ng/mL and propionyl-L-carnitine within a scope of 4-320 ng/mL in a sample.

Further, the sample is subject serum.

Further, the quantitative determination results are obtained by serving a Cell Free Amino Acid Mix 20 AA, O-acetyl-L-carnitine hydrochloride (N-methyl-D3) and lysophosphatidyl choline (20:0) (eicosacarbonyl-12,12,13,13-D4) as isotope internal standards for analysis.

Further, the integrated biomarker system further includes a model built by the machine learning method.

Further, the machine learning method is eXtreme Gradient Boosting (XGBoost).

Compared with the prior art, the present invention has the following advantages:

The present invention discloses an integrated biomarker system for evaluating a risk of IFG and T2DM for the first time. The integrated biomarker system for IFG and T2DM of subject serum sample established by the present invention contains a correlative biomarker group on a biological network path to reflect the overall metabolic characteristics information of IFG and T2DM and to avoid that characteristic information of a disease cannot be reflected integrally and completely due to single or separate analysis of a biomarker. The quantitative-based integrated biomarker system provided by the present invention is from a clinical real world, and has multi-center clinical study and stronger representativeness, thus improving the potential clinical application value of biomarkers of diseases. Further, the targeted quantitative evaluation and detection method established in this present invention has high sensitivity, strong specificity, good reproducibility, a small amount of detection samples, and simple operation.

### BRIEF DESCRIPTION OF THE DRAWINGS

FIG. 1A is a chromatogram showing a selective reaction monitoring (SRM) of L-glutamine, FIG. 1B is an SRM chromatogram of L-valine, FIG. 1C is an SRM chromatogram of L-leucine, FIG. 1D is an SRM chromatogram of L-lysine, FIG. 1E is an SRM chromatogram of L-proline, and FIG. 1F is an SRM chromatogram of L-phenylalanine; the three columns (left, center and right) of each of FIGS. 1A-1F respectively represent results of a solvent blank, standards and serum samples;

FIG. 2A is an SRM chromatogram of L-arginine, FIG. 2B is an SRM chromatogram of L-glutamic acid, FIG. 2C is an SRM chromatogram of L-isoleucine, FIG. 2D is an SRM chromatogram of L-methionine, FIG. 2E is an SRM chromatogram of L-carnitine, and FIG. 2F is an SRM chromatogram of acetyl-L-carnitine; the three columns (left, center and right) of each of FIGS. 2A-2F respectively represent results of a solvent blank, standards and serum samples;

FIG. 3A is an SRM chromatogram of lysophosphatidyl choline (LPC, P-16:0), FIG. 3B is an SRM chromatogram of LPC (17:0), FIG. 3C is an SRM chromatogram of LPC (14:0), and FIG. 3D is an SRM chromatogram of propionyl-L-carnitine; the three columns (left, center and right) of each of FIGS. 3A-3D respectively represent results of a solvent blank, standards and serum samples;

FIGS. 4A-4P are violin plots of 16 metabolite concentrations in subject serum sample; FIG. 4A shows the plot for L-glutamine, FIG. 4B shows the plot for L-valine, FIG. 4C shows the plot for L-leucine, FIG. 4D shows the plot for L-lysine, FIG. 4E shows the plot for L-proline, FIG. 4F shows the plot for L-phenylalanine, FIG. 4G shows the plot for L-arginine, FIG. 4H shows the plot for L-glutamic acid, FIG. 4I shows the plot for L-isoleucine, FIG. 4J shows the plot for L-methionine, FIG. 4K shows the plot for L-carnitine, FIG. 4L shows the plot for acetyl-L-carnitine, FIG. 4M shows the plot for lysophosphatidyl choline (LPC, P-16:0), FIG. 4N shows the plot for LPC (17:0), FIG. 4O shows the plot for LPC (14:0), and FIG. 4P shows the plot for propionyl-L-carnitine;

FIG. 5 is a performance result diagram for the classification and diagnosis of subject serum sample via 16 metabolites;

FIG. 6 shows a graphical result of areas under the curve of the 16 metabolites in three machine learning models;

FIG. 7 is an incremental feature selection curve of the 16 metabolites based on Gini impurity, mutual information and analysis of variance of an XGBoost model;

FIG. 8 is an ordering diagram showing Gini impurity of the 16 metabolites in subject serum sample;

FIG. 9 shows a graphical result of areas under the curve of the preferred 10 metabolites by three machine learning models;

FIG. 10 shows an integrated biomarker system for NGT (normal glucose tolerance), IFG, T2DM and hyperlipidemia;

FIG. 11 is a schematic diagram showing a result of a representative sample 1 evaluated by the integrated biomarker system (NGT);

FIG. 12 is a schematic diagram showing a result of a representative sample 2 evaluated by the integrated biomarker system (IFG);

FIG. 13 is a schematic diagram showing a result of a representative sample 3 evaluated by the integrated biomarker system (T2DM);

FIG. 14 is a schematic diagram showing a result of a representative sample 4 evaluated by the integrated biomarker system (hyperlipidemia).

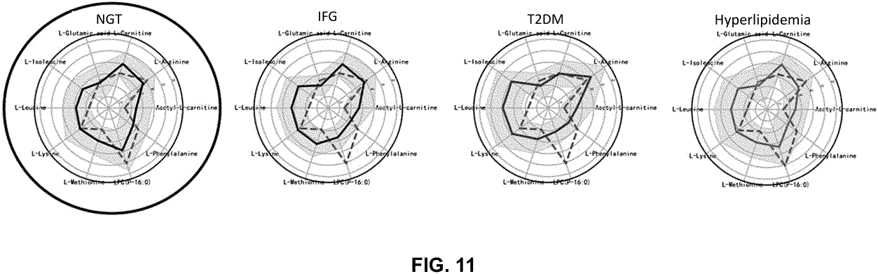

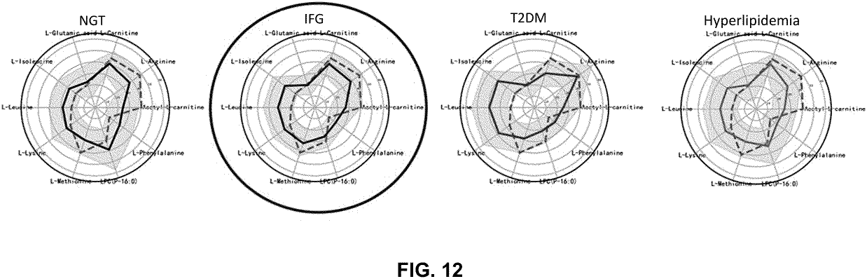

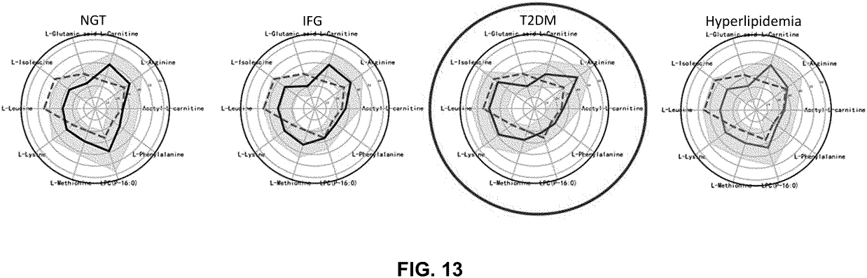

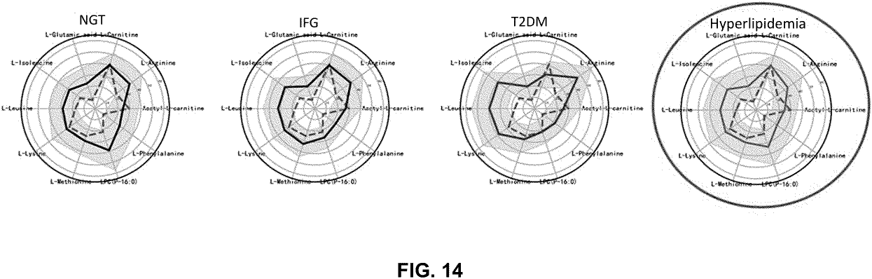

LPC in FIGS. 11-14 is lysophosphatidyl choline.

### DETAILED DESCRIPTION OF THE EMBODIMENTS

To further describe the technical means and results taken by the present invention to achieve the predetermined goals of the present invention, preferred examples will be used to describe the detailed embodiments, technical solution and features of the present application specifically below. Specific features, structures or characteristics in a plurality of examples in the description below may be combined in any appropriate form.

Main materials and sources selected and used in the following examples of the present invention are respectively as follows:

The L-glutamine (batch No.: V900419), L-valine (batch No.: 94619), L-leucine (batch No.: 61819), L-lysine (batch No.: 23128), L-proline (batch No.: 81709), L-phenylalanine (batch No.: 852465P), L-arginine (batch No.: 11009-25G-F), L-glutamic acid (batch No.: 95436), L-isoleucine (batch No.: I2752), L-methionine (batch No.: 64319-25G-F), lysophosphatidyl choline (LPC (P-16:0)) (batch No.: 852464P), LPC (17:0) (batch No.: 855676P), LPC (14:0) (batch No.: 855575P) and propionyl-L-carnitine (batch No.: 91275) used in the analysis are purchased from Sigma-Aldrich; L-carnitine (batch No.: DRE-C11045500) is purchased from Beijing J&K Scientific Co., Ltd.; acetyl-L-carnitine hydrochloride (batch No.: DST190510-049) is purchased from Chengdu Desite Biotechnology Co., Ltd.; the isotope Cell Free Amino Acid Mix (20 AA) (U-D, 98%)) (batch No.: DLM-6819-PK), O-acetyl-L-carnitine hydrochloride (N-methyl-D3, 98%) (batch No.: DLM-754-0.05) and LPC (20:0) (eicosacarbonyl-12,12,13,13-D4, 98%) (batch No.: DLM-10520-0.001) are purchased from Cambridge Isotope Laboratories; ammonium acetate (batch No.: E057G140) is purchased from CNW Technologies GmbH; ultra-performance liquid chromatography (UPLC) Quadrupole-Orbitrap high-resolution and precise mass spectrometry (Thermo Fisher Scientific, Q-Exactive); UPLC triple quadrupole mass spectrometer (Thermo Fisher Scientific, TSQ-Altis); refrigerated micro-centrifuge (Thermo Fisher Scientific, Heraeus Fresco 17); multi-purpose vortex mixer (Scientific Industries, Vortex Genie 2); 5 mL serum separation hose (Becton, Dickinson and Company, 367955); and reversed phase column (Waters, ACQUITY BEH C₁₈ and ACQUITY BEH HILIC).

### Example I: Sample Collection

The sample for the integrated biomarker system in the present invention is from subject serum.

Subjects were recruited from 5 clinical centers of Beijing, Zhengzhou and Kaifeng and serum samples were collected. To eliminate diet disturbance, the subject serum samples were together collected at 7:00-9:00 a.m. after overnight fasting. Peripheral venous blood of the subjects was collected with 5 mL serum separation hoses. After standing for 30 min, peripheral venous blood was centrifuged for 10 min at 1510 g with a refrigerated high-speed centrifugal machine at a condition of 4° C., then 200 µL supernatant were taken and subpackaged into 1.5 mL labelled EP tubes, and stored in a -80° C. refrigerator before analysis. Finally, 1132 parts of serum samples were totally collected and then used for the subsequent analysis.

### Example II: Preparation of Standard Curve Working Solution and Quality Control (QC) Samples

A proper amount of standards L-glutamine, L-valine, L-leucine, L-lysine, L-proline, L-isoleucine, L-methionine, L-phenylalanine, L-arginine, L-glutamic acid, L-carnitine and Cell Free Amino Acid Mix (20 AA) were weighed and respectively placed in 10 mL volumetric flasks, then 10% methanol aqueous solution was added for dissolving and fixing a constant volume to prepare into a stock solution, where L-glutamine has a concentration of 4000 µg/mL; L-valine, L-leucine, L-lysine, L-proline, L-isoleucine and L-methionine have a concentration of 2000 µg/mL; L-phenylalanine, L-arginine, L-glutamic acid and L-carnitine have a concentration of 1000 µg/mL; and 20 AA has a concentration of 1000 µg/mL.

A proper amount of LPC (P-16:0), LPC (17:0), LPC (14:0), propionyl-L-carnitine, LPC (20:0) (eicosacarbonyl-12,12,13,13-D4, 98%) (LPC (20:0)-d4) were weighed, and acetonitrile aqueous solution (1:1, v:v) was added for dissolving and fixing a constant volume to prepare into a stock solution in which LPC (P-16:0), LPC (17:0), LPC (14:0), propionyl-L-carnitine and LPC (20:0)-d4 had a concentration of 100 µg/mL.

A proper amount of acetyl-L-carnitine and O-acetyl-L-carnitine hydrochloride (N-methyl-D3, 98%) (acetyl-L-carnitine-d3) were weighed, and 4% hydrochloric acid aqueous solution was added for dissolving and fixing a constant volume to prepare into a stock solution in which L-acetylcarnitine had a concentration of 100 µg/mL and acetyl-L-carnitine-d3 had a concentration of 100 µg/mL.

The above prepared stock solutions were put and stored in a 4° C. refrigerator for further use.

A proper amount of the above prepared stock solution of 20 AA, acetyl-L-carnitine-d3 and LPC (20:0)-d4 were precisely absorbed and put in a 500 mL volumetric flask, and acetonitrile-methanol (3:1, v:v) solution was added for dissolving and fixing a constant volume to prepare into an acetonitrile-methanol protein precipitant working solution containing internal standards 20 AA, acetyl-L-carnitine-d3 and LPC (20:0)-d4 respectively having a concentration of 10 µg/mL, 500 ng/mL and 25 ng/mL.

Because human blank serum is hardly obtained conventionally, 1x phosphate buffered solution is used to substitute blank serum as a blank control. A proper amount of the stock solution of standards was absorbed, and 1x phosphate buffer solution was added for stepwise dilution to prepare into 7 concentration levels of standard curve working solutions; three concentrations (low, middle and high) of QC samples (LQC, MQC and HQC) were set and used for the subsequent quantitative analysis for the samples. Concentrations of the standard curve working solutions and QC samples are as shown in Table 1.

TABLE 1

Linearity concentrations of the standard curve working solution and QC samples
Concentration level of the standard curve working solution (ng/mL)

| | | | | | | | | |
|---|---|---|---|---|---|---|---|---|
| Metabolite | 1 | 2 (LQC) | 3 | 4 | 5 (MQC) | 6 | 7 | HQC |
| L-glutamine | 2000 | 4000 | 10000 | 40000 | 80000 | 120000 | 200000 | 160000 |
| L-valine | 1200 | 2400 | 6000 | 24000 | 48000 | 72000 | 120000 | 96000 |
| L-leucine | 1000 | 2000 | 5000 | 20000 | 40000 | 60000 | 100000 | 80000 |
| L-lysine | 800 | 1600 | 4000 | 16000 | 32000 | 48000 | 80000 | 64000 |
| L-proline | 800 | 1600 | 4000 | 16000 | 32000 | 48000 | 80000 | 64000 |
| L-phenylalanine | 500 | 1000 | 2500 | 10000 | 20000 | 30000 | 50000 | 40000 |
| L-arginine | 500 | 1000 | 2500 | 10000 | 20000 | 30000 | 50000 | 40000 |
| L-glutamic acid | 500 | 1000 | 2500 | 10000 | 20000 | 30000 | 50000 | 40000 |
| L-isoleucine | 300 | 600 | 1500 | 6000 | 12000 | 18000 | 30000 | 24000 |
| L-methionine | 250 | 500 | 1250 | 5000 | 10000 | 15000 | 25000 | 20000 |
| L-carnitine | 200 | 400 | 1000 | 4000 | 8000 | 12000 | 20000 | 16000 |
| Acetyl-L-carnitine | 80 | 160 | 400 | 1600 | 3200 | 4800 | 8000 | 6400 |
| LPC (P-16:0) | 60 | 120 | 300 | 1200 | 2400 | 3600 | 6000 | 4800 |
| LPC (17:0) | 60 | 120 | 300 | 1200 | 2400 | 3600 | 6000 | 4800 |
| LPC (14:0) | 40 | 80 | 200 | 800 | 1600 | 2400 | 4000 | 3200 |
| Propionyl-L-carnitine | 4 | 8 | 20 | 80 | 160 | 240 | 400 | 320 |

### Example III: Quantitative Analysis of the Sample

Sample pretreatment: 10 µL of the prepared standard curve working solution or QC sample was precisely absorbed and put to a 1.5 mL centrifuge tube, and 90 µL serum samples were added for dilution, and mixed well by vortex for 1 min; 300 µL acetonitrile-methanol protein precipitant working solution was added and mixed well by vortex for 5 min; then mixture was centrifuged for 10 min at 16,200 g with a condition of 4° C., then supernatant was taken and used for the subsequent analysis.

Chromatographic conditions: a Waters ACQUITY BEH HILIC (100 mm × 2.1 mm, 1.7 µm) chromatographic column was used; a mobile phase A was 0.1% formic acid aqueous solution containing 20 mmol/L ammonium acetate, a mobile phase B was acetonitrile containing 0.1% formic acid; injection volume was 3 µL, flow rate was 0.30 mL/min, and column temperature was 40° C.; liquid phase elution procedure: the initial mobile phase B was 95% and kept for 2.0 min, and linearly dropped to 60% at 4.0 min, after keeping for 6.0 min, linearly increased to 95% within 0.2 min and kept for 1.8 min; the whole analysis operation time was 12 min.

Mass spectrometry conditions: electrospray ionization mode was a positive ion mode (ESI⁺); and the monitoring mode was selective reaction monitoring. Spray voltage was 3.5 kV, collision gas was high-purity nitrogen, auxiliary gas had a flow rate of 17 L/min; ion transmission tube had a temperature of 325° C., and the evaporator had a temperature of 320° C. Sheath gas had a flow rate of 20 L/min.

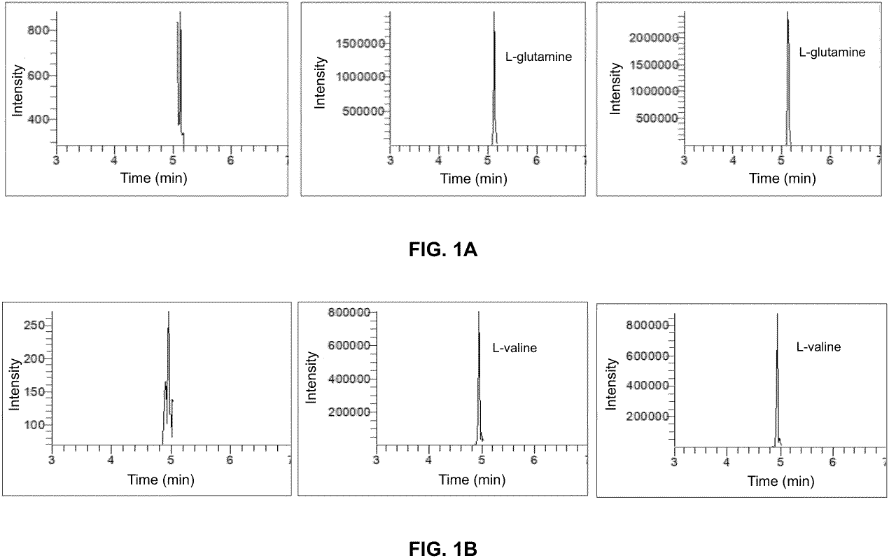

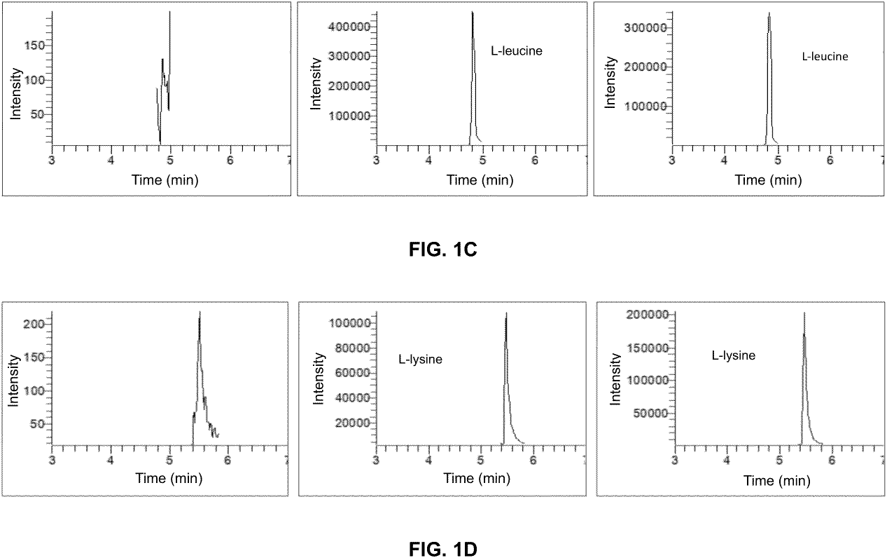

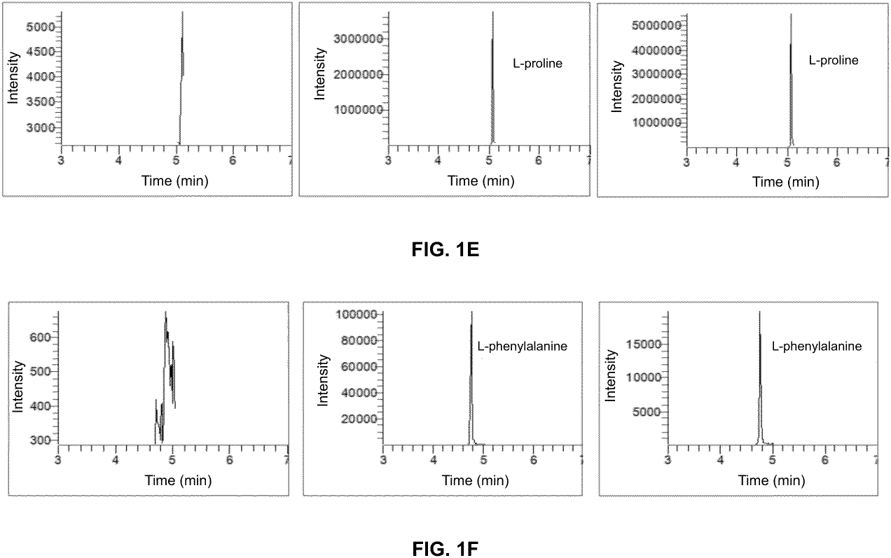

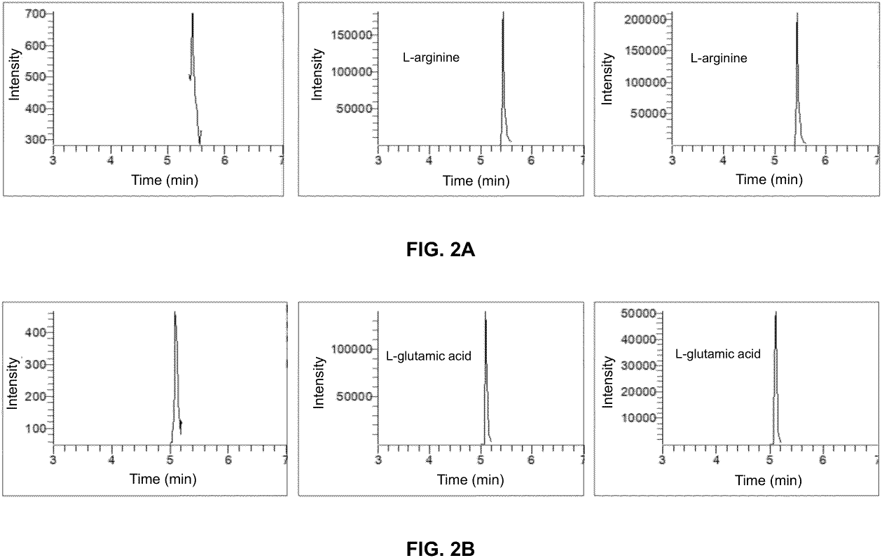

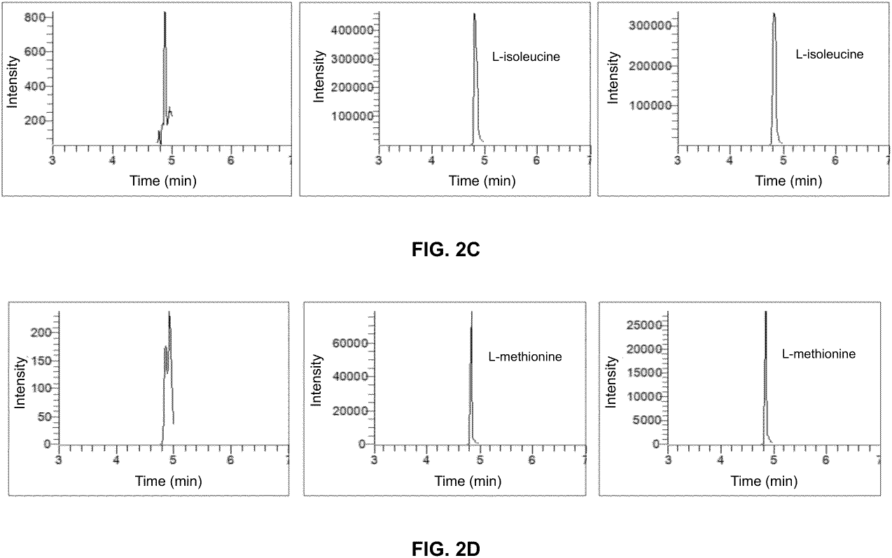

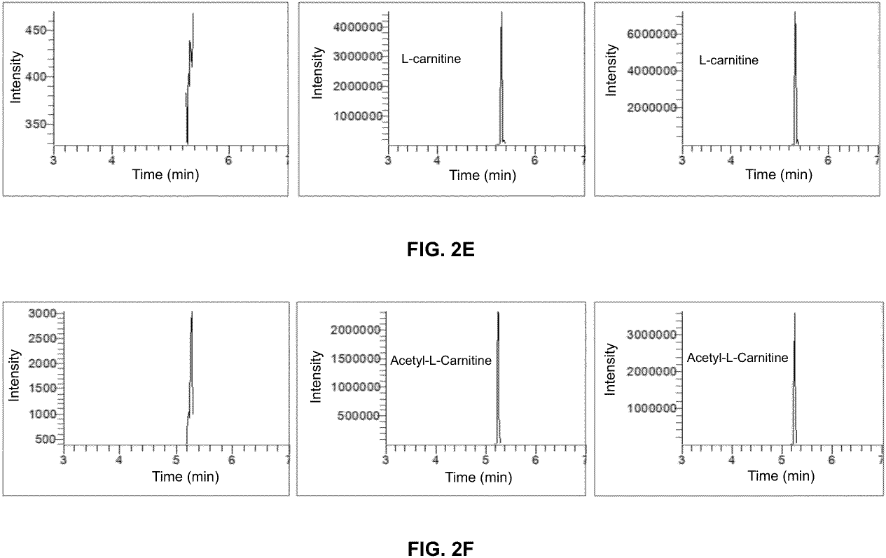

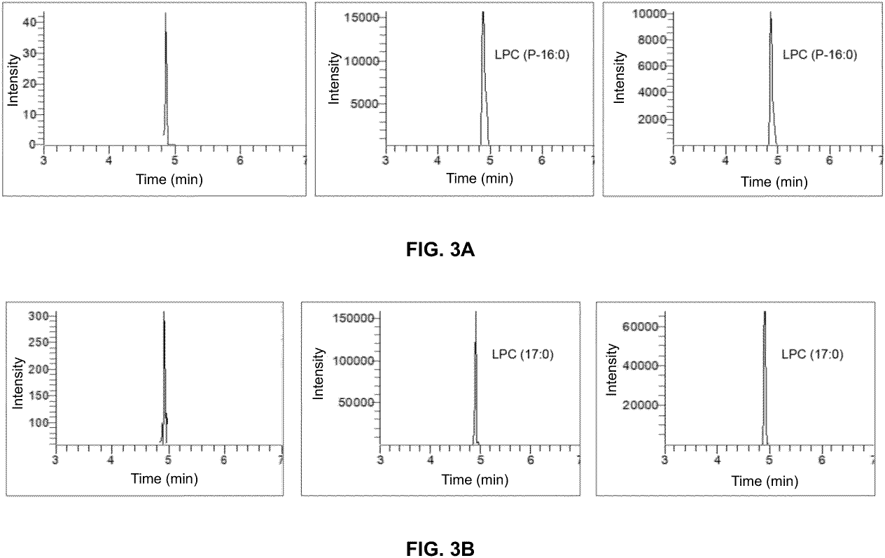

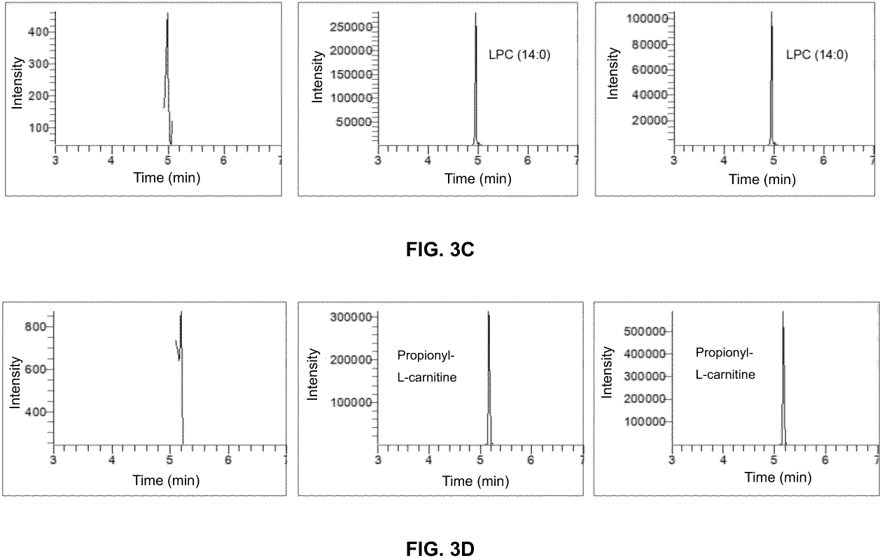

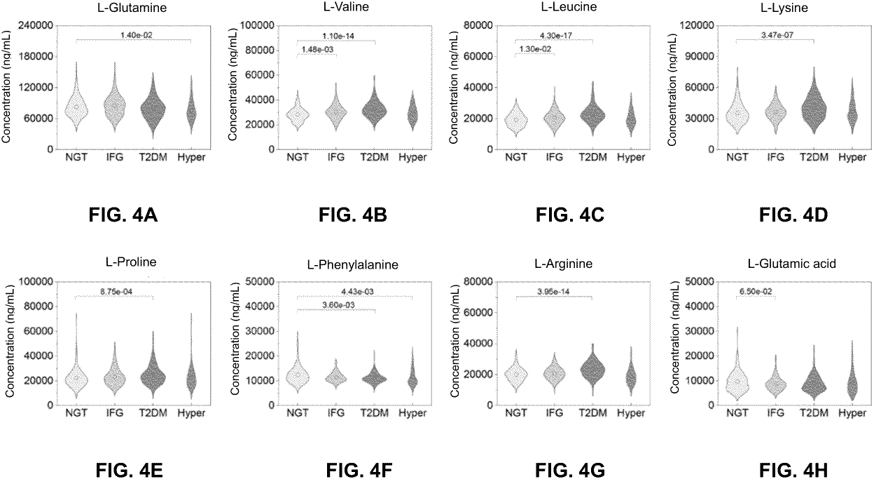

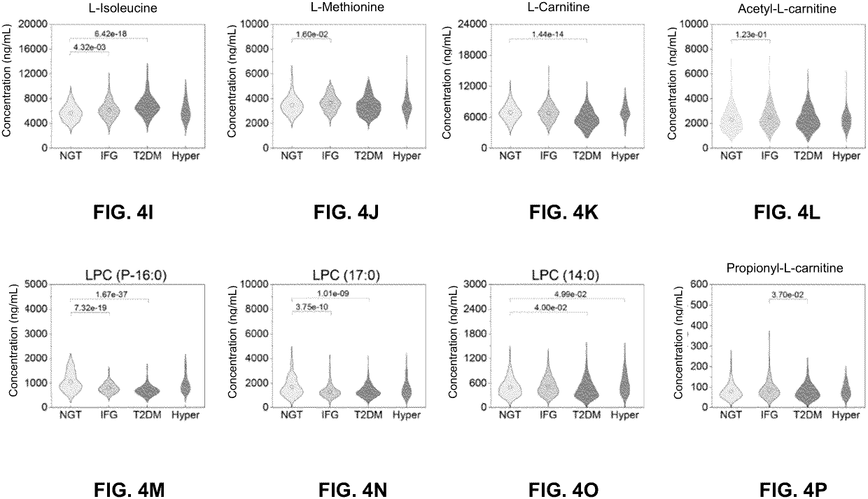

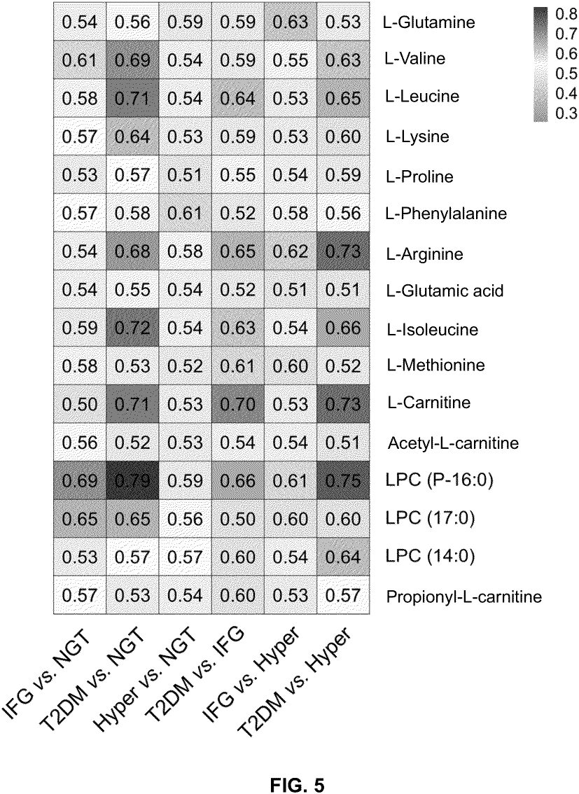

6 parts of serum samples obtained in Example I were drawn randomly and pretreated by the above pretreatment method; meanwhile, 6 parts of the pretreated blank controls and 6 parts of the pretreated 1x phosphate buffer solution were prepared, then the above samples were analyzed. The results are shown in FIGS. 1-3, indicating that each endogenous substance had no interference on analytes and isotope internal standards in the measured serum samples, and there was a good degree of separation between the to-be-analyzed metabolites and isotope internal standards.

Results of the lower limit of quantitation (LLOQ), limit of detection (LOD), linearity and concentration range and precision are shown in Table 2. The metabolites show good linearity (correlation coefficient R value is greater than 0.99) within the prepared concentration range; the intra-day precision relative standard deviation (RSD) of the surveyed 6 batches of LQC, MQC and HQC is 2.08%-11.87%; and inter-day precision RSD is 1.68%-11.23%.

TABLE 2

Results of LLOQ and LOD, linearity, concentration range and precision

| Metabolite | Linearity range (ng/mL) | Coefficient (R²) | LLOQ (ng/mL) | LOD (ng/mL) | The selected isotope internal standards | Precision (RSD%) | | | | | |
|---|---|---|---|---|---|---|---|---|---|---|---|
| | | | | | | Intra-day | | | Inter-day | | |
| | | | | | | LQC | MQC | HQC | LQC | MQC | HQC |
| L-glutamine | 2000-200000 | 0.9944 | 2000 | 600 | L-glutamic acid- d5 | 5.48 | 6.56 | 5.45 | 6.75 | 8.39 | 5.46 |
| L-valine | 1200-120000 | 0.9920 | 1200 | 360 | L-valine-d8 | 2.38 | 4.12 | 4.77 | 2.14 | 1.85 | 1.68 |
| L-leucine | 1000-100000 | 0.9938 | 1000 | 300 | L-leucine-d10 | 2.69 | 3.31 | 6.02 | 6.42 | 2.24 | 3.31 |
| L-lysine | 800-80000 | 0.9958 | 800 | 240 | L-arginine-d7 | 4.77 | 3.58 | 5.42 | 3.92 | 3.85 | 4.67 |
| L-proline | 800-80000 | 0.9984 | 800 | 240 | L-proline-d7 | 3.52 | 2.61 | 5.18 | 2.87 | 2.73 | 2.21 |
| L-phenylalanine | 500-50000 | 0.996 | 500 | 150 | L-phenylalanine-d8 | 7.52 | 4.26 | 2.10 | 9.70 | 3.71 | 2.96 |
| L-arginine | 500-50000 | 0.996 | 500 | 150 | L-arginine-d7 | 3.04 | 4.14 | 2.31 | 1.68 | 2.20 | 3.50 |
| L-glutamic acid | 500-50000 | 0.9971 | 500 | 150 | L-glutamic acid-d5 | 4.43 | 7.08 | 5.49 | 3.5 | 2.02 | 2.20 |
| L-isoleucine | 300-30000 | 0.9904 | 300 | 90 | L-leucine-d10 | 4.76 | 3.27 | 6.01 | 5.57 | 1.74 | 3.27 |
| L-methionine | 250-25000 | 0.9972 | 250 | 75 | L-methionine-d5+d3 | 11.87 | 3.62 | 7.35 | 8.78 | 4.02 | 5.34 |
| L-carnitine | 200-20000 | 0.9973 | 200 | 60 | Acetyl-L-carnitine-d3 | 2.08 | 3.78 | 4.91 | 3.75 | 2.79 | 1.98 |
| Acetyl-L-carnitine | 80-8000 | 0.9954 | 80 | 24 | Acetyl-L-carnitine-d3 | 6.02 | 3.23 | 7.23 | 4.68 | 4.4 | 1.98 |
| LPC (P-16:0) | 60-6000 | 0.9935 | 60 | 18 | LPC (20:0)-d4 | 6.21 | 5.19 | 8.9 | 10.64 | 3.86 | 3.62 |
| LPC (17:0) | 60-6000 | 0.9947 | 60 | 18 | LPC (20:0)-d4 | 3.65 | 7.06 | 3.70 | 5.11 | 4.33 | 3.68 |
| LPC (14:0) | 40-4000 | 0.9959 | 40 | 12 | LPC (20:0)-d4 | 6.66 | 5.48 | 10.42 | 3.69 | 4.58 | 4.69 |
| Propionyl-L-carnitine | 4-400 | 0.9848 | 4 | 1.2 | Acetyl-L-carnitine-d3 | 2.60 | 4.88 | 7.39 | 4.20 | 2.50 | 11.23 |

Results of the intra-day accuracy, extraction recovery rate and matrix effect are shown in Table 3; the intra-day accuracy relative error (RE) of the LQC, MQC and HQC is -13.33%-13.72%; the inter-day accuracy RE is -13.30%-13.18%; the average extraction recovery rate of the 16 metabolites at LQC and HQC sample concentrations is 68.68%-129.87%; the average matrix effect is 74.54%-142.93%.

TABLE 3

Results of the accuracy, extraction recovery rate and matrix effect

| Metabolite | Accuracy (RE%) | | | | | | Average extraction recovery rate (%) | | Average matrix effect (%) | |
|---|---|---|---|---|---|---|---|---|---|---|
| | Put for 24 h at 10° C. | | | Put for 24 h at 4° C. | | | | | | |
| | LQC | MQC | HQC | LQC | MQC | HQC | LQC | HQC | LQC | HQC |
| L-glutamine | -8.52 | 2.64 | -2.31 | -11.76 | -6.00 | -2.06 | 114.64 | 99.43 | 101.08 | 110.01 |
| L-valine | 3.09 | 13.49 | 10.88 | -12.11 | 3.91 | 10.55 | 97.04 | 96.00 | 102.89 | 107.73 |
| L-leucine | -4.52 | 5.60 | 6.18 | -13.30 | 0.29 | 6.49 | 97.55 | 96.02 | 86.89 | 94.7 |
| L-lysine | 9.20 | 13.03 | 10.42 | -8.03 | 7.75 | -3.00 | 99.42 | 99.61 | 94.33 | 94.79 |
| L-proline | 13.72 | 8.25 | 8.90 | -5.29 | 1.56 | 5.55 | 99.31 | 97.75 | 103.19 | 105.83 |
| L-phenylalanine | 2.36 | 12.07 | 11.94 | 7.72 | -3.01 | 9.82 | 116.58 | 100.71 | 112.64 | 123.98 |
| L-arginine | 0.25 | 12.96 | 12.6 | -10.12 | 5.10 | 13.18 | 100.75 | 98.51 | 99.87 | 104.81 |
| L-glutamic acid | -12.07 | 5.76 | 9.48 | -4.07 | -7.67 | 4.07 | 129.87 | 97.34 | 83.55 | 108.43 |
| L-isoleucine | -2.44 | 5.44 | 6.15 | -11.81 | -0.15 | 5.89 | 98.79 | 95.97 | 82.19 | 94.27 |
| L-methionine | -1.61 | 9.17 | 12.94 | 10.83 | -0.59 | 10.36 | 89.42 | 92.79 | 98.92 | 107.60 |
| L-carnitine | -13.19 | -11.44 | -11.97 | 0.88 | 7.22 | 9.15 | 98.34 | 96.73 | 91.34 | 92.38 |
| Acetyl-L-carnitine | -13.33 | -6.39 | 9.69 | -8.33 | 0.42 | 12.93 | 96.54 | 94.40 | 79.37 | 84.33 |
| LPC (P-16:0) | -7.13 | 5.77 | 11.84 | 2.50 | 1.70 | 6.07 | 106.98 | 97.05 | 74.54 | 135.17 |
| LPC (17:0) | 10.26 | 8.64 | 7.75 | -9.65 | 4.79 | 10.81 | 87.76 | 93.25 | 128.89 | 142.51 |
| LPC (14:0) | 2.61 | 11.62 | 1.06 | -10.61 | 4.44 | 8.60 | 82.73 | 68.68 | 132.25 | 142.93 |
| Propionyl-L-carnitine | -12.13 | -5.94 | 13.35 | 0.27 | -12.07 | -9.55 | 95.77 | 93.37 | 106.17 | 128.11 |

Results of the stability are shown in Table 4. When the metabolites were put to an automatic sampler for 24 h at the concentrations of LQC, MQC and HQC, the stability RSD is 0.85%-9.78%; when the metabolites were put in a 4° C. refrigerator for 24 h, the stability RSD is 0.97%-10.20%; when the metabolites were put in a 5-fold dilution condition, the RSD is 0.60%-5.72%, indicating that the content determination of metabolites in the serum samples was free of influence under the 5-fold dilution condition. Through test, the residuals in the residual effect bank samples of the 16 metabolites were less than 20% of the LLOQ.

TABLE 4

Results of stability and dilution effect
Stability (RSD%)

| Metabolite | Put for 24 h at 10° C. | | | Put for 24 h at 4° C. | | | Dilution effect |
|---|---|---|---|---|---|---|---|
| | LQC | MQC | HQC | LQC | MQC | HQC | (RSD%) |
| L-glutamine | 0.85 | 1.94 | 1.70 | 2.67 | 1.89 | 1.60 | 1.32 |
| L-valine | 5.51 | 2.86 | 3.12 | 4.68 | 1.03 | 4.41 | 0.60 |
| L-leucine | 3.96 | 3.39 | 6.89 | 2.54 | 2.74 | 3.07 | 2.31 |
| L-lysine | 2.61 | 1.67 | 2.28 | 2.61 | 2.44 | 1.62 | 3.00 |
| L-proline | 2.78 | 2.14 | 1.7 | 2.43 | 2.38 | 1.82 | 3.09 |
| L-phenylalanine | 5.34 | 4.08 | 2.31 | 10.2 | 3.99 | 3.97 | 1.84 |
| L-arginine | 1.89 | 2.46 | 5.35 | 1.17 | 2.01 | 1.80 | 1.28 |
| L-glutamic acid | 2.32 | 1.90 | 2.81 | 4.67 | 1.73 | 1.84 | 2.64 |
| L-isoleucine | 3.54 | 2.05 | 4.44 | 2.49 | 1.12 | 4.61 | 1.75 |
| L-methionine | 2.63 | 6.65 | 6.26 | 2.88 | 5.67 | 5.10 | 3.44 |
| L-carnitine | 6.23 | 3.18 | 2.26 | 4.93 | 2.85 | 0.97 | 1.71 |
| Acetyl-L-carnitine | 6.29 | 4.85 | 5.15 | 7.88 | 2.64 | 3.13 | 2.25 |
| LPC (P-16:0) | 9.78 | 4.38 | 1.79 | 6.71 | 3.64 | 4.92 | 3.77 |
| LPC (17:0) | 4.12 | 3.27 | 2.38 | 3.74 | 4.74 | 4.92 | 3.52 |
| LPC (14:0) | 3.81 | 3.09 | 2.74 | 3.96 | 5.99 | 6.26 | 5.72 |
| Propionyl-L-carnitine | 5.47 | 8.68 | 7.90 | 2.56 | 1.83 | 5.75 | 3.81 |

The above results prove that the selectivity, LLOQ and LOD, linearity and concentration range, precision and accuracy, extraction recovery rate and matrix effect, stability, dilution effect and residual effect of the targeted detection method used in this present invention accord with the requirements of the quantitative analysis method of serum biological samples.

### Example IV: Establishment and Application of the Integrated Biomarker System

The method in Example III was used to determine the 1132 parts of samples collected in Example I. NGT, IFG, T2DM and hyperlipidemia samples were used to build a model.

The sample data set was randomly divided into a training set and a test set by a 70-30 holdout method; the training set (232 parts of NGT, 314 parts of IFG, 230 parts of T2DM and 96 parts of hyperlipidemia) was used for training the model; and the test set (80 parts of NGT, 97 parts of IFG, 113 parts of T2DM and 50 parts of hyperlipidemia) was used for testing the model.

After data was extracted by TraceFinder software, the metabolite difference was analyzed with Kruskal-Wallis, and the difference among multiple groups was adjusted by Bonferroni correction; Origin 2019 software was used to draw the targeted metabolite content of the training set and the test set. As shown in Table 4, the results show that the serum concentration of the 16 targeted metabolites in the training set and the test set has significant difference. A single metabolite was subjected to receiver operator characteristic curve analysis, and area under the curve (AUC) was used for performance evaluation. The results are shown in Table 5, and a single metabolite has poor evaluation performance to the four types of samples. In terms of systems biology, it is of higher value to serve a plurality of associated metabolites as a biomarker for the evaluation of disease risk. Therefore, machine learning methods were used to establish an evaluation model of IFG and T2DM integrated biomarker system with 16 targeted metabolites.

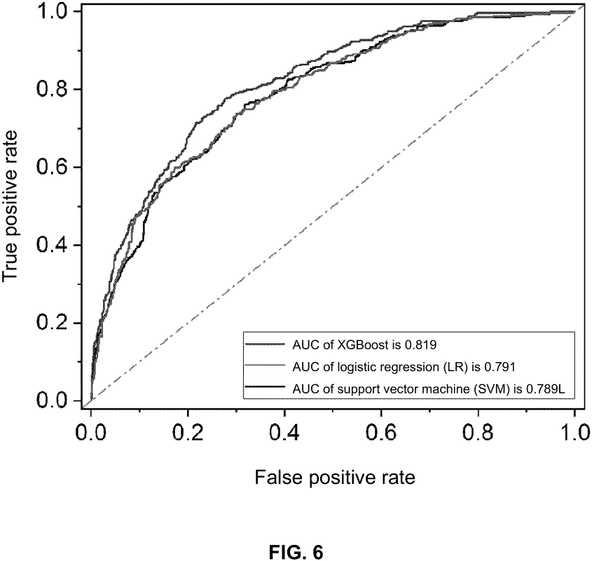

Further, to screen a suitable method to build the evaluation model of IFG and T2DM integrated biomarker system, AUC served as an evaluation index in the test set to evaluate three machine learning methods (eXtreme Gradient Boosting (XGBoost), Logistic Regression (LR) and Support Vector Machine (SVM). The results are shown in FIG. 6. As can be seen in FIG. 6, in terms of AUC value, the XGBoost model has optimal distinguishing performance to four types of samples, namely, NGT, IFG, T2DM and hyperlipidemia (XGBoost model has an AUC value of 0.819, LR model has an AUC value of 0.791, and SVM model has an AUC value of 0.789). Therefore, XGBoost was selected to build the integrated biomarker system model.

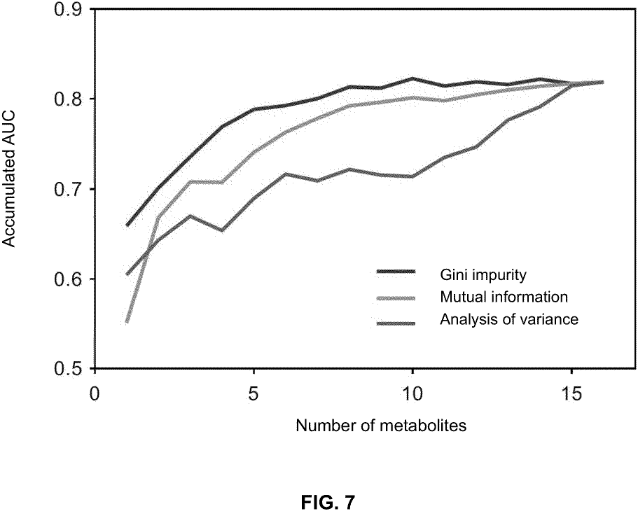

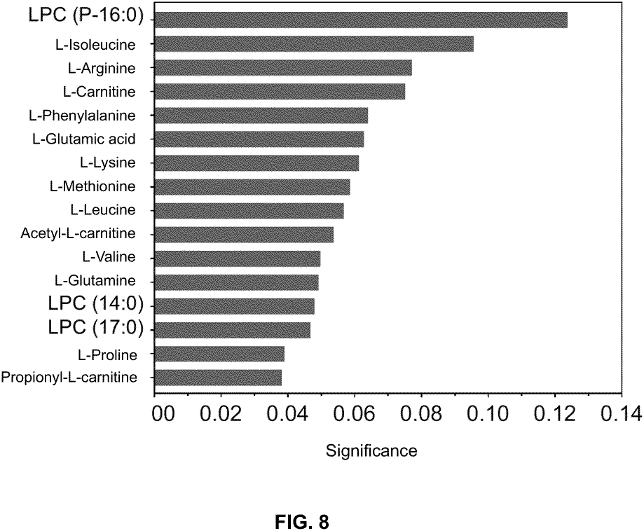

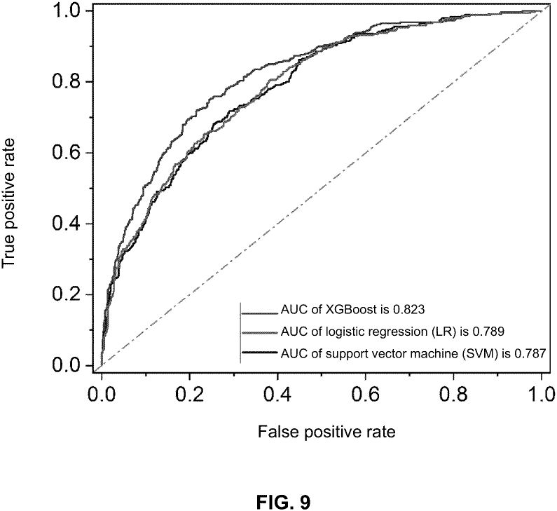

To improve the specificity and sensitivity of the evaluation model, the significance of metabolites was ordered by Gini impurity, mutual information and analysis of variance; and the optimal metabolite subset was determined by an incremental feature selection strategy. The results are shown in FIGS. 7-8; in the XGBoost model based on Gini impurity, when the number of major metabolites increases to 11, the model does not show better performance. Therefore, as a preferred solution, ordered by Gini impurity, the former 10 metabolites, namely, LPC (P-16:0), L-isoleucine, L-arginine, L-carnitine, L-phenylalanine, L-glutamic acid, L-lysine, L-methionine, L-leucine and acetyl-L-carnitine were selected to constitute an integrated biomarker system. As shown in FIG. 9, the XGBoost model has an AUC value of 0.823. Obviously, the evaluation performance of the model built by 10 metabolites in the XGBoost model is higher than that of 16 metabolites.

The test set was used to evaluate the performance of the model; AUC, accuracy, sensitivity, specificity, precision and F1 score were used for evaluation. The results are shown in Table 5.

TABLE 5

Performance evaluation of the integrated biomarker system

| | AUC | Accuracy | Sensitivity | Specificity | Precision | F1 score |
|---|---|---|---|---|---|---|
| IFG vs. NGT | 0.804 | 0.701 | 0.713 | 0.690 | 0.667 | 0.689 |
| T2DM vs. NGT | 0.936 | 0.852 | 0.879 | 0.823 | 0.847 | 0.862 |
| Hyperlipidemia vs. NGT | 0.689 | 0.703 | 0.541 | 0.762 | 0.455 | 0.494 |
| T2DM vs. IFG | 0.823 | 0.749 | 0.782 | 0.710 | 0.761 | 0.771 |
| IFG vs. hyperlipidemia | 0.754 | 0.739 | 0.625 | 0.786 | 0.543 | 0.581 |
| T2DM vs. hyperlipidemia | 0.937 | 0.889 | 0.786 | 0.786 | 0.805 | 0.795 |
| NGT vs. IFG vs. T2DM | 0.835 | 0.666 | 0.659 | 0.822 | 0.662 | 0.671 |
| NGT vs. IFG vs.T2DM vs. hyperlipidemia | 0.823 | 0.576 | 0.552 | 0.863 | 0.531 | 0.530 |

It can be seen from the data of Table 5 that the model has an accuracy of 85% to the identification of 2DM and NGT, and respectively has an accuracy of 75% and 89% to the identification of T2DM and IFG, T2DM and hyperlipidemia. Therefore, the model may be used for evaluating the risk of NGT, IFG, T2DM and hyperlipidemia.

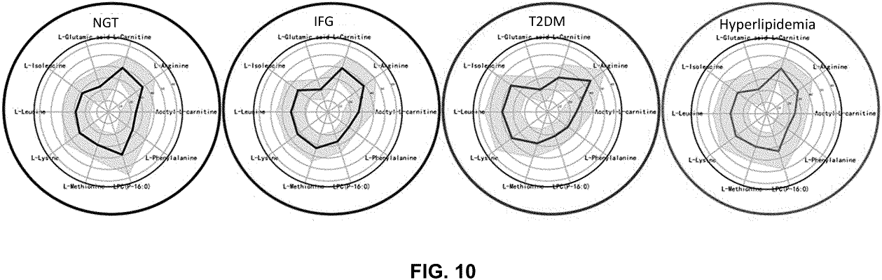

To visualize the integrated biomarker system of IFG and T2DM, a formula was used to normalize the original data: value of the biomarker after normalization(B₍ᵢ₎) =(concentration of the biomarker before normalization (B_{(c)}) -minimum concentration of the biomarker before normalization (B₍ₘᵢₙ₎))/(maximum concentration of the biomarker before normalization (B₍ₘₐₓ₎)) -minimum concentration of the biomarker before normalization (B₍ₘᵢₙ₎)) × 100; after normalization, B₍ᵢ₎ mean value ± standard deviation (mean ± SD), and mean ± SD was used for plotting. The results are shown in FIG. 10; the full line represents the mean value of the concentration of the 10 metabolites after normalization in the four types of samples; gray area represents mean ± SD, and dotted line represents the concentration of the 10 metabolites of unknown samples. The integrated biomarker system established on the basis of XGBoost may be interpreted as that the unknown sample is evaluated as the one having the highest assessed value in the four types.

Furthermore, a schematic diagram having representative evaluation results of samples is represented as well, as shown in FIGS. 11-14. The sample 1 has a greater risk of suffering from NGT (the assessed value is 0.795 in the NGT group); the sample 2 has a greater risk of suffering from IFG (the assessed value is 0.676 in the IFG group); the sample 3 has a greater risk of suffering from T2DM (the assessed value is 0.597 in the T2DM group); and the sample 4 has a greater risk of suffering from hyperlipidemia (the assessed value is 0.702 in the hyperlipidemia group).

What is described above are merely preferred embodiments of the present invention, but the protection scope of the present invention is not limited thereto. Any equivalent replacement or change made by a person skilled in the art based on the technical solution and improvement concept of the present invention within the technical scope disclosed herein shall be covered within the protection scope of the present invention.

What is claimed is:

1. An integrated biomarker system for evaluating a risk of impaired fasting glucose (IFG) and type 2 diabetes mellitus (T2DM), wherein the integrated biomarker system comprises quantitative determination results of L-glutamine within a scope of 2,000-16,0000 ng/mL, L-valine within a scope of 1,200-96,000 ng/mL, L-leucine within a scope of 1,000-8,0000 ng/mL, L-lysine within a scope of 800-64,000 ng/mL, L-proline within a scope of 800-64,000 ng/mL, L-phenylalanine within a scope of 500-40,000 ng/mL, L-arginine within a scope of 500-40,000 ng/mL, L-glutamic acid within a scope of 500-40,000 ng/mL, L-isoleucine within a scope of 300-24,000 ng/mL, L-methionine within a scope of 250-20,000 ng/mL, L-carnitine within a scope of 200-16,000 ng/mL, acetyl-L-carnitine within a scope of 80-6,400 ng/mL, lysophosphatidyl choline (LPC (P-16:0)) within a scope of 60-4,800 ng/mL, LPC (17:0) within a scope of 60-4,800 ng/mL, LPC (14:0) within a scope of 40-3,200 ng/mL, and propionyl-L-carnitine within a scope of 4-320 ng/mL in a sample.

2. The integrated biomarker system according to claim 1, wherein the sample is subject serum.

3. The integrated biomarker system according to claim 1, wherein the quantitative determination results are obtained by serving a Cell Free Amino Acid Mix 20 AA, O-acetyl-L-carnitine hydrochloride (N-methyl-D3) and lysophosphatidyl choline (20:0) (eicosacarbonyl-12,12,13,13-D4) as isotope internal standards for analysis.

4. The integrated biomarker system according to claim 1, wherein the integrated biomarker system further comprises a model built by a machine learning method.

5. The integrated biomarker system according to claim 4, wherein the machine learning method is eXtreme Gradient Boosting (XGBoost).
